(19) United States
(12) Patent Application Publication
(10) Pub. No.: US 2005/0182433 A1
(43) Pub. Date: Aug. 18, 2005

(54) Device for receiving and cutting specimen tissue

(76) Inventor: Nady E. Nady, Skillman, NJ (US)

Correspondence Address: Gregory C. Houghton, Esq.;Mathews, Collins, Shepherd & McKay, P.A., Suite 306, 100 Thanet Circle, Princeton, NJ, 08540, US

(21) Appl. No.: 10/778,567

(22) Filed: Feb. 13, 2004

(51) Int. Cl. A61B 17/32

(52) U.S. Cl. 606/170

(57) ABSTRACT

A device useful for receiving and cutting specimen tissue during surgical procedures.

### BACKGROUD OF THE INVENTION

1. Field of the Invention

The present invention relates to a device for receiving and cutting specimen tissue during surgical procedures.

2. Related Art

Laparoscopic surgery has gained increasing importance within both gynecological and general surgeries because it avoids the need to make large surgical incisions to access the surgical area during surgical procedures. In laparoscopic surgery small puncture orifices are made in a body cavity, such as the abdominal or thoracic cavity, through which laparoscopic instruments are inserted to assist in an array of surgical procedures, which include the removal of specimen tissue (organ or surgically removed tissue). The abdomen or a body cavity is inflated by gas, which increases the volume considerably allowing survey and accessibility during laparoscopic surgery. When the specimen tissue is small in size it may be extracted through one of the laparoscopic instruments however, if the specimen is large in size it is either necessary to open the abdomen by making an incision therein, thus reducing the advantages of the laparoscopic surgical procedure or to cut (morcellate) the specimen. Instruments for this purpose (morcellators) are well known and understood by those skilled in the art as well as the problems associated with their use (See U.S. Pat. No. 5,443,472). The most considerable problem associated with current morcellators is damage that may be made to surrounding organs (for example, blood vessels) or the areas surrounding the specimen tissue being cut since there is no safe barrier between the morcellator and the surrounding areas of the body cavity.

It is therefore desirable to provide a device to effectively receive and safely cut a specimen tissue without damaging surrounding tissue areas.

### SUMMARY OF THE INVENTION

The present invention provides a device for receiving and cutting specimen tissue from a body cavity that includes an outer structure element and an inner rod adapted to be received through the outer structure element. The outer structure element can be cylindrical in shape. The inner rod has a forward end and a rearward end. Also, the device includes at least two flexible elements coupled to the forward end of the inner rod, wherein each of the flexible elements has a cutting surface. The flexible elements define an entrapment area for receiving the specimen tissue. The flexible elements that define the entrapment area cut the specimen tissue received therein inside the body cavity as the inner rod is withdrawn through the outer structure element bringing the specimen tissue toward said the structure element.

An alternate embodiment of the invention provides a device for receiving and cutting specimen tissue from a body cavity that includes an outer structure element having at least two flexible elements coupled to the outer structure element. The outer structure element can be cylindrical in shape. Each of the flexible elements has a cutting surface and the flexible elements define an entrapment area for receiving the specimen tissue. The device also includes an inner rod adapted to be received through the outer structure element. The inner rod has a forward end with a gripping device and a rearward end. The inner rod is introduced through the specimen tissue and withdrawn through the outer structure element, to bring the specimen tissue into the entrapment area. Also, as the inner rod is withdrawn, the gripping device of the inner rod grips the flexible elements that define the entrapment area to bring the flexible elements in contact with the specimen tissue in order to cut the specimen tissue received therein inside the body cavity as the inner rod is withdrawn through the outer structure element.

Another embodiment of the present invention provides a device for receiving and cutting a specimen tissue from a body cavity which includes an outer structure element having at least two outer flexible elements coupled to the outer structure element and each of the outer flexible elements having a cutting surface and an attachment means. The device includes a hollow inner rod adapted to be received through the outer structure element. Also, the device includes an inner flexible element adapted to be received through the hollow inner rod. The inner flexible element has a forward end with at least two gripping devices and a rearward end. The gripping devices are adapted to attach to the attachment means of the outer flexible elements to define an entrapment area for receiving the specimen tissue.

When the hollow inner rod with the inner flexible element is introduced through the specimen tissue and withdrawn through the outer structure element, the gripping devices of the inner flexible element attach to the attachment means of the outer flexible elements. This brings the specimen tissue into the entrapment area. Also, as the hollow inner rod and the inner flexible element are withdrawn through the outer structure this brings the cutting surface of the outer flexible elements in contact with the specimen tissue to cut the specimen tissue inside the body cavity.

### BRIEF DESCRIPTION OF THE DRAWINGS

The advantage, nature, and various additional features of the invention will appear more fully upon consideration of the illustrative embodiments now to be described in detail in connection with accompanying drawings wherein:

FIG. 1 is a schematic diagram of a device for receiving and cutting specimen tissue during surgical procedures.

FIG. 2 is a schematic diagram of a device for receiving and cutting specimen tissue in accordance to an alternate embodiment of the invention.

FIGS. 3A and 3B are schematic diagrams of a device for receiving and cutting specimen tissue in accordance to an alternate embodiment of the invention.

It should be understood that the drawings are for purposes of illustrating the concept of the invention and are not necessarily to scale.

### DETAILED DESCRIPTION OF THE INVENTION

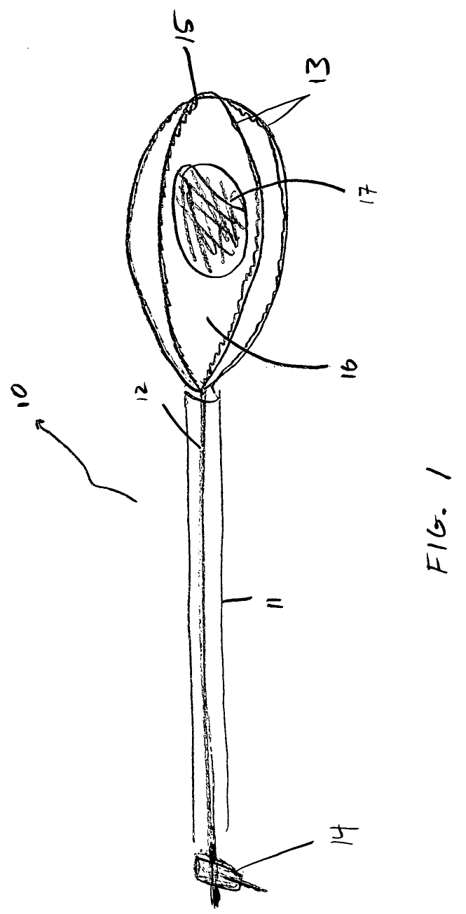

Referring to the drawings wherein like reference numerals identify similar or like elements throughout the several views and initially to FIG. 1 there is shown an exemplary embodiment of the present invention. FIG. 1 shows device 10 in accordance with the invention. Device 10 receives and cuts (morcellates) specimen tissue during surgical procedures.

Device 10 includes outer structure element 11 primarily cylindrical in shape and inner rod 12. Inner rod 12 is adapted to be received through outer structure element 11. Inner rod 12 has a forward end portion with flexible elements 13 and a rearward end portion with adjustment means 14 at the opposite end. Each of flexible elements 13 has cutting surface 15. Flexible elements 13 define entrapment area 16 for receiving specimen tissue 17.

Flexible elements 13 can be adjusted by adjustment means 14. Adjustment means 14 changes size of entrapment area 16. Adjustment means 14 can hold and/or lock flexible elements 13 in a fixed profile or position enabling entrapment area 16 to be held in a fixed profile or position. Adjustment means are well known to those skilled in the art and include but are not limited to a latch-type system used by pliers, clippers, ratchets, and/or plastic ties.

During surgical procedures, device 10 is introduced into a body cavity (not shown). When device 10 is inserted into the body cavity, inner rod 12 with flexible elements 13 is retracted inside outer element structure 11 as to protect surrounding tissue areas of the surgical procedure area from flexible elements 13. Once inside the body cavity, inner rod 12 is pushed through outer structure element 11 and flexible elements 13 are introduced into the body cavity to receive specimen tissue 17. When specimen tissue 17 is received in entrapment area 16, inner rod 12 is withdrawn through outer structure element 11. Withdrawing inner rod 12 through outer structure element 11 decreases the size of entrapment area 16 size and brings specimen tissue 17 toward outer structure element 11. Outer structure element 11 enables specimen tissue 17 to be held in a static position within entrapment area 16 and enables flexible elements 13 to cut specimen tissue 17 inside the body cavity when inner rod 12 is withdrawn through outer structure element 11. Adjustment means 14 enables inner rod 12 to be withdrawn through outer structure element 11 enabling entrapment area 16 to be decreased by bringing cutting surface 15 of each of flexible elements 13 toward each other enabling flexible elements 13 to cut specimen tissue 17.

Device 10 can be made of metal or plastic. Device 10 can be disposable.

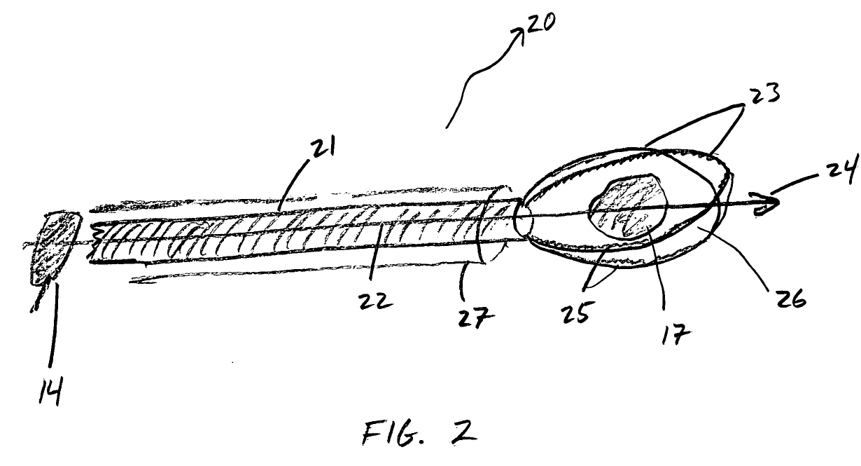

FIG. 2 shows device 20 in accordance with an alternate embodiment of the invention. Device 20 receives and cuts (morcellates) specimen tissue during surgical procedures.

Device 20 includes outer structure element 21 and inner rod 22. Inner rod 22 is adapted to be received through outer structure element 21. Outer structure element 21 has a forward end portion with flexible elements 23. Inner rod 22 has a forward end portion with gripping device 24 and a rearward end portion with adjustment means 14 at the opposite end. Each of flexible elements 23 has cutting surface 25. Flexible elements 23 define entrapment area 26 for receiving specimen tissue 17. Gripping device 24 grips flexible elements 23 when inner rod 22 is withdrawn through outer structure element 21 by adjustment means 14 enabling a change in size of entrapment area 26. Device 20 can have protective shield 27. Protective shield 27 covers outer structure element 21 and flexible elements 23. Protective shield 27 protects the surrounding tissue of the surgical procedure area from flexible elements 23 when device 20 is introduced into a body cavity (not shown). In an alternate embodiment, Flexible elements 23 can be retracted inside outer structure element 21 when device 20 is introduced into the body cavity.

Protective shield 27 can be made of an elastomeric material or plastic, for example polyurethane, poly(vinyl chloride) or any other suitable material. Protective shield 26 can be replaceable and/or disposable.

Device 20 can be made of metal or plastic and can be disposable.

Once inside the body cavity, flexible elements 23 are introduced into the body cavity to receive specimen tissue 17. When specimen tissue 17 is received in entrapment area 26, inner rod 22 with gripping device 24 is pushed through specimen tissue 17. Inner rod 22 is withdrawn through outer structure element 21 enabling gripping device 24 to grip flexible elements 23. Withdrawing inner rod 22 through outer structure element 21 brings specimen tissue 17 toward outer structure element 21. Outer structure element 21 enables specimen tissue 17 to be held in a static position within entrapment area 26 and enables flexible elements 23 to cut specimen tissue 17 inside the body cavity when inner rod 22 is withdrawn through outer structure element 21. Adjustment means 14 enables inner rod 22 with gripping device 24 to be withdrawn through outer structure element 21 enabling gripping device to grip flexible elements 23 as inner rod 22 is withdrawn through outer structure element 21 decreasing entrapment area 26 by bringing cutting surface 25 of each of flexible elements 23 toward each other enabling flexible elements 23 to cut specimen tissue 17.

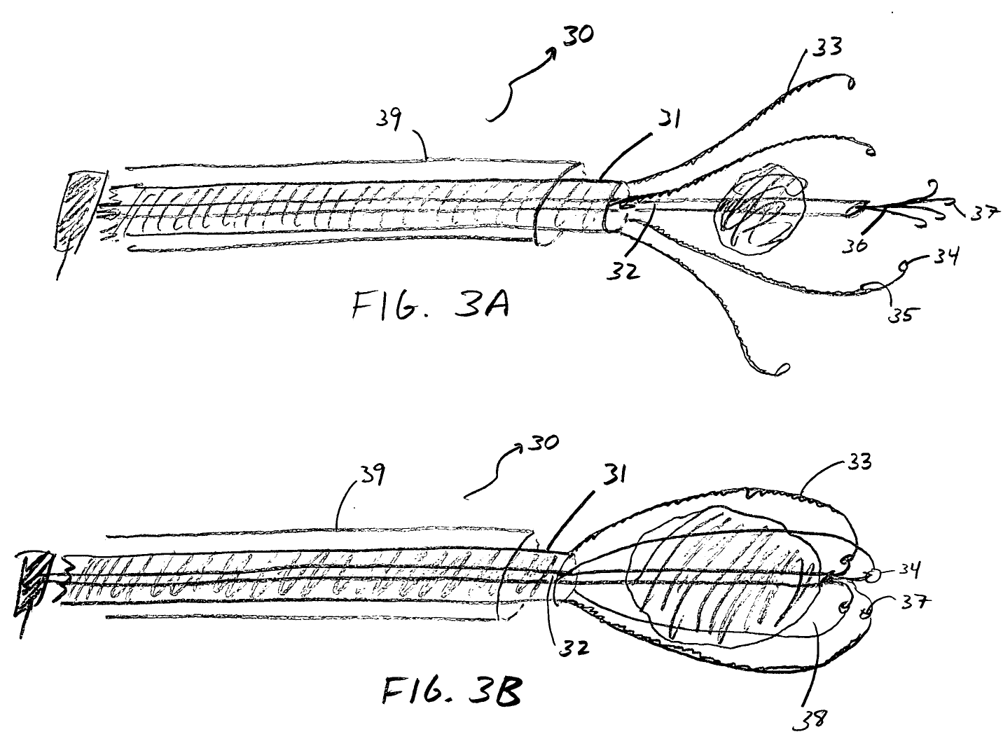

FIG. 3A and FIG. 3B show device 30 in accordance to an alternate embodiment of the invention. Device 30 receives and cuts (morcellates) specimen tissue during surgical procedures.

Device 30 includes outer structure element 31 and hollow inner rod 32. Hollow inner rod 32 is adapted to be received through outer structure element 31. Outer structure element 31 has a forward end portion with outer flexible elements 33. Each of outer flexible elements 33 has an attachment means 34 and cutting surface 35. Hollow inner rod 32 is adapted to receive inner flexible elements 36. Each of inner flexible elements 36 has a forward end portion with gripping device 37 and a rearward end portion with adjustment means 14 at the opposite end. Outer flexible elements 33 and inner flexible elements 36 define entrapment area 38 for receiving specimen tissue 17. Gripping device 37 grips attachment means 34 of outer flexible elements 33 when inner rod 32 is withdrawn through outer structure element 31 by adjustment means 14 enabling a change in size of entrapment area 38.

Device 30 can have protective shield 39. Protective shield 39 covers outer structure element 31 and outer flexible elements 33. Protective shield 39 protects the surrounding tissue of the surgical procedure area from outer flexible elements 33 when device 30 is introduced into a body cavity (not shown). In an alternate embodiment, Outer flexible elements 33 can be retracted inside outer structure element 31 when device 30 is introduced into the body cavity.

Protective shield 39 can be made of an elastomeric material or plastic, for example polyurethane, poly(vinyl chloride) or any other suitable material. Protective shield 39 can be replaceable and/or disposable.

Device 30 can be made of metal or plastic and can be disposable.

Once inside the body cavity, outer flexible elements 33 are introduced into the body cavity to receive specimen tissue 17.

When specimen tissue 17 is to be received in entrapment area 38, hollow inner rod 32 with inner flexible elements 36 received therein is pushed through specimen tissue 17. Hollow inner rod 32 and inner flexible elements 36 are withdrawn through outer structure element 31 enabling gripping device 37 of each of inner flexible elements 36 to grip each of outer flexible elements 33. Withdrawing hollow inner rod 32 and inner flexible elements 36 through outer structure element 31 brings specimen tissue 17 toward outer structure element 31. Outer structure element 31 enables specimen tissue 17 to be held in a static position within entrapment area 38 and enables inner flexible elements 36 to cut specimen tissue 17 inside the body cavity when hollow inner rod 32 and inner flexible elements 36 are withdrawn through outer structure element 31. Adjustment means 14 enables inner flexible elements 36 with gripping device 37 to be withdrawn through outer structure element 31 enabling gripping device 37 of each of inner flexible elements 36 to grip attachment means 34 of outer flexible elements 33 as inner flexible elements 36 are withdrawn through outer structure element 31 decreasing entrapment area 38 by bringing cutting surface 35 of each of outer flexible elements 33 toward each other enabling outer flexible elements 33 to cut specimen tissue 17.

Specimen tissue 17 can be introduced in entrapment areas 16, 26, and 38 by means of an additional laparoscopic instrument (not shown).

Specimen tissue 17 once cut by devices 10, 20, and 30 are collected and removed from the body cavity by means of an additional laparoscopic instrument (not shown).

Cutting surfaces 15, 25, and 35 can be just be the corresponding flexible elements 13, 23, and, 33 themselves or the flexible elements can have serrated edges. Also, as an exemplary embodiment, flexible elements 13, 23, and 33 and/or cutting surfaces 15, 25, and 35 can be connected to an electrosurgical unit that enables flexible elements 13, 23, and 33 and/or cutting surfaces 15, 25, and 35 to use high frequency electrical current to cut specimen 17. Electrosurgical units are well understood by those skilled in the art and include but are not limited by electrosurgical units commonly used in laparoscopic pelvic surgery such as the ELMED ESU 70 DIGITAL B Electrosurgical unit.

It is to be understood that the above-described embodiments are illustrative of only a few of the many possible specific embodiments which can represent applications of the principles of the invention. Numerous and varied other arrangements can be readily devised in accordance with these principles by those skilled in the art without departing from the spirit and scope of the invention.

1. A device for receiving and cutting a specimen tissue from a body cavity, said device comprising:
an outer structure element;
an inner rod adapted to be received through said outer structure element, said inner rod having a forward end and a rearward end; and
at least two flexible elements coupled to said forward end of said inner rod, each of said flexible elements having a cutting surface wherein said flexible elements define an entrapment area for receiving the specimen tissue;
said flexible elements of said entrapment area cut the specimen tissue received therein as said inner rod is withdrawn through said outer structure element bringing said specimen tissue toward said outer structure element.

2. The device according to claim 1 further comprising an adjustment means coupled to said rearward end of said inner rod wherein said adjustment means adjusts the size of said entrapment area.

3. The device according to claim 2 wherein said adjustment means holds said entrapment area in a fixed position.

4. The device according to claim 1 wherein said outer structure element is primarily cylindrical in shape.

5. A method for receiving and cutting a specimen tissue from a body cavity during surgical procedures, said method comprising the steps of:
introducing a device into the body cavity, said device comprising an outer structure element; an inner rod adapted to be received through said outer structure element, said inner rod having a forward end and a rearward end; and at least two flexible elements coupled to said forward end of said inner rod, each of said flexible elements having a cutting surface wherein said flexible elements define an entrapment area for receiving the specimen tissue;
positioning a specimen tissue in said entrapment area; and
withdrawing said inner rod through said outer structure element to enable said specimen tissue to be brought toward said outer structure element within said entrapment area and bring each of said cutting surfaces of said flexible elements in contact with said specimen tissue for cutting said specimen tissue.

6. The method according to claim 5 wherein said device further comprises an adjustment means coupled to said rearward end of said inner rod wherein said adjustment means adjust the size of said entrapment area.

7. The method according to claim 6 wherein said adjustment means holds said entrapment area in a fixed position.

8. A device for receiving and cutting a specimen tissue from a body cavity, said device comprising:
an outer structure element;
at least two flexible elements coupled to said outer structure element, each of said flexible elements having a cutting surface wherein said flexible elements define an entrapment area for receiving the specimen tissue; and
an inner rod adapted to be received through said outer structure element, said inner rod having a forward end with a gripping device and a rearward end;
said inner rod is introduced through the specimen tissue and as said inner rod is withdrawn through said outer structure element, the specimen tissue is brought into said entrapment area, said gripping device of said inner rod grips said flexible elements of said entrapment area to cut the specimen tissue received therein as said inner rod is withdrawn through said outer structure element bringing the specimen tissue toward said outer structure element.

9. The device according to claim 8 further comprising an adjustment means coupled to said rearward end of said inner rod wherein said adjustment means adjust the size of said entrapment area.

10. The device according to claim 8 wherein said outer structure element is primarily cylindrical in shape.

11. The device according to claim 9 wherein said adjustment means holds said entrapment area in a fixed position.

12. The device according to claim 8 further comprising a protective shield.

13. The device according to claim 12 wherein said protective shield is made of an elastomeric material.

14. A method for receiving and cutting a specimen tissue within a body cavity during a surgical procedure, said method comprising the steps of:
introducing a device into the body cavity, said device comprising an outer structure element; at least two flexible elements coupled to said outer structure element, each of said flexible elements having a cutting surface wherein said flexible elements define an entrapment area for receiving the specimen tissue; and an inner rod adapted to be received through said outer structure element, said inner rod having a forward end with a gripping device and a rearward end;
positioning a specimen tissue in said entrapment area;
introducing said inner rod with said gripping device through said specimen tissue; and
withdrawing said inner rod through said outer structure element to enable said specimen tissue to be brought toward said outer structure element within said entrapment area and enables said gripping device to grip said flexible elements to bring each of said cutting surfaces of said flexible elements in contact with said specimen tissue cutting said specimen tissue.

15. The method according to claim 14 wherein said device further comprises an adjustment means coupled to said rearward end of said inner rod wherein said adjustment means adjust the size of said entrapment area.

16. The method according to claim 15 wherein said adjustment means holds said entrapment area in a fixed position.

17. The method according to claim 14 wherein said device further comprises a protective shield.

18. The method according to claim 17 wherein said protective shield is made of an elastomeric material.

19. A device for receiving and cutting a specimen tissue from a body cavity, said device comprising:
an outer structure element;
at least two outer flexible elements coupled to said outer structure element, each of said outer flexible elements having a cutting surface and an attachment means;
a hollow inner rod adapted to be received through said outer structure element;
an inner flexible element adapted to be received through said hollow inner rod, said inner flexible element having a forward end with at least two gripping devices and a rearward end, said gripping device adapted to attach to said attachment means of said outer flexible elements to define an entrapment area for receiving the specimen tissue;
said hollow inner rod with said inner flexible element is introduced through the specimen tissue and as said inner flexible element is withdrawn through said outer structure element, said gripping device of said inner flexible element attaches to said attachment means of said outer flexible elements, the specimen tissue is brought into said entrapment area to cut the specimen tissue received therein as said inner flexible element is withdrawn through said outer structure element bringing the specimen tissue toward said outer structure element.

20. The device according to claim 19 wherein said outer structure element is primarily cylindrical in shape.

21. The device according to claim 19 further comprising an adjustment means coupled to said rearward end of said inner rod wherein said adjustment means adjusts the size of said entrapment area.

22. The device according to claim 21 wherein said adjustment means holds said entrapment area in a fixed position.

23. The device according to claim 19 further comprising a protective shield.

24. The device according to claim 23 wherein said protective shield is made of an elastomeric material.

25. A method for receiving and cutting a specimen tissue from a body cavity during a surgical procedure, said method comprising the steps of:
introducing a device into the body cavity, said device comprising an outer structure element; at least two outer flexible elements coupled to said outer structure element, each of said outer flexible elements having a cutting surface and an attachment means; a hollow inner rod adapted to be received through said outer structure element; an inner flexible element adapted to be received through said hollow inner rod, said inner flexible element having a forward end with at least two gripping devices and a rearward end, said gripping device adapted to attach to said attachment means of said outer flexible elements to define an entrapment area for receiving the specimen tissue;
introducing said hollow inner rod with said inner flexible elements received therein through said specimen tissue;
withdrawing said hollow inner rod and said inner flexible elements through said outer structure element to enable said specimen tissue to be brought toward said outer structure element and enables said gripping device of each of said inner flexible elements to grip said attachment means of each of said outer flexible elements to form said entrapment area surrounding said specimen tissue and to bring each of said cutting surfaces of said flexible elements in contact with said specimen tissue cutting said specimen tissue.

26. The device according to claim 25 further comprising an adjustment means coupled to said rearward end of said inner rod wherein said adjustment means adjusts the size of said entrapment area.

27. The device according to claim 26 wherein said adjustment means holds said entrapment area in a fixed position.

28. The device according to claim 25 further comprising a protective shield.

29. The device according to claim 28 wherein said protective shield is made of an elastomeric material.
